(19) United States
(12) Patent Application Publication
(10) Pub. No.: US 2025/0064835 A1
(43) Pub. Date: Feb. 27, 2025

(54) A COMPOSITION FOR TREATING OCULAR INFECTIONS AND A METHOD OF PREPARATION THEREOF

(71) Applicants: KHALID KHAN, New Delhi (IN); SHRUTI KAUSHIK, New Delhi (IN); CHANDER SHEKHAR, New Delhi (IN)

(72) Inventors: CHANDER SHEKHAR, New Delhi (IN); FARHAAN JALEES AHMAD, New Delhi (IN)

(21) Appl. No.: 18/723,593

(22) PCT Filed: Dec. 22, 2022
(86) PCT No.: PCT/IB2022/062676

§ 371 Date: Jun. 24, 2024

(30) Foreign Application Priority Data
Dec. 22, 2021 (IN) 202111048327

(51) Int. Cl. A61K 31/593; A61K 9/107; A61K 31/7036; A61K 38/14; A61K 47/10; A61K 47/14; A61K 47/26

(52) U.S. Cl. CPC A61K 31/593; A61K 9/1075; A61K 31/7036; A61K 38/14; A61K 47/10; A61K 47/14; A61K 47/26

(57) ABSTRACT

The present invention provides a composition for treating ocular infections. The composition of the present invention comprises of vitamin D nanoemulsion, an antimicrobial agent and a pharmaceutically accepted salt in desired amount for the treatment of ocular infections with enhanced healing. The invention further provides a method of preparation of the vitamin D nanoemulsion as well as a method of preparation of said composition.

### FIELD OF THE INVENTION

The present invention relates to a composition for treating ocular infections and a method of preparation thereof. More particularly, the present invention relates to a composition comprising vitamin D nanoemulsion and an antimicrobial agent for the management of ocular infections with enhanced healing and a method of preparation of the same.

### BACKGROUND OF THE INVENTION

The eye is susceptible to bacterial and parasitic infections arising from both traumatic and non-traumatic related events. Infections are a concern after ocular surgery and precautions are correspondingly taken to prevent the onset of infection. However, even without the invasive trauma of a surgical procedure, infections in the eyelids, conjunctiva, cornea and other ocular tissues can occur. Treating infections in ocular tissues can be challenging and/or problematic because of the difficulty in delivering an antibiotic to the affected tissue. In general, ocular infections are treated by local injection, systemic administration or topical application of an antibiotic. The route of administration depends on the antibiotic selected, the location of the infection and the type of infection. The simple and direct approach of topically applying the antibiotic to the exterior of the eye has several benefits, including the avoidance of side effects and reduced chance of developing resistant strains of bacteria as compared to systemic administration. However, for a variety of reasons, many antibiotics are not amenable or suitable for topical application to the eye.

Vitamin D is a multifunctional hormone that is known to play a significant role in a variety of biological functions in addition to its traditional role in regulating calcium homeostasis. Vitamin D is now recognized to have many diverse functions including effects on immune regulation, proliferation, differentiation, apoptosis and angiogenesis. Vitamin D directly or indirectly regulates up to 5% of the human genome, or over 900 different genes. There are a large number of studies demonstrating that adequate vitamin D levels are important in maintaining health and show that vitamin D is able to be utilized at local tissue sites. In the eye, there is increasing evidence of the association between disease and vitamin D.

In addition, vitamin D receptor (VDR) is almost ubiquitously expressed. The enzyme necessary for conversion of vitamin D to its functional metabolite has been identified in a number of cell types, which are able to utilize circulating vitamin D to form the biologically active hormone. Extrarenal activation, expression and gene influence suggest that vitamin D function is widespread, with pleiotropic effects within the tissue microenvironment where it is activated.

In a recent study, the inhibitory effect of vitamin D3 against the expression of MHC class antigens in order to suppress any rejection was observed during the keratoplasty suggesting that vitamin D3 may serve to control the rejection of transplantation of cornea. Further, active vitamin D is known to control the cellular activity of, for instance, corneal epithelial cells, keratocytes, tissues in the anterior ocular region, inflammatory cells and lens epithelial cells, can maintain the optical transparency and normal refractive power of the anterior ocular region as well as the normal intraocular pressure and can prevent any reduction in the visual functions.

JP2017518332A discloses an ophthalmic composition for administration to a patient's eye for the treatment of an eye infection. The ophthalmic composition contains an amount of ε-polylysine (εPL) effective for the treatment or control or prevention of an eye infection.

U.S. Pat. No. 7,056,893B2 discloses a method for treating or preventing infections using stabilized aqueous compositions containing azalide antibiotics, which do not have to be reconstituted prior to use. Azalide antibiotic compositions especially adapted to treat infections of the eye and surrounding tissues.

U.S. Pat. No. 9,919,012B2 discloses compositions and methods that effectively support innate immunity and/or disperse pathogenic biofilms using readily available, nontoxic, natural substances, while supporting restoration of normal microbiotic homeostasis. The subject matter also discloses anti-biofilm compositions comprising one or more probiotic organisms, anti-microbial honey and other ingredients such as prebiotic compounds, other hive products, green tea derivatives, other plant derivatives and vitamin D3.

Chander Shekhar (2019) disclosed a composition comprising vitamin D3 granules impregnated with Tobramycin or Tobramycin and Vancomycin for the treatment of infected open wounds in patients of diabetes mellitus, with necrotizing fasciitis and post-operative infections as well as treating difficult cases of infected wounds sustained in road accidents.

However, none of the above cited document discloses effective healing of the ocular infections which the present invention focuses on by employing the components such as that of the instant invention which results in enhanced healing in case of ocular infections.

### OBJECT AND SUMMARY OF THE INVENTION

The main object of the present invention is to provide a composition for the management of ocular infections.

Another object of the present invention is to provide a composition with anti-microbial property for the management of ocular infections.

Yet another object of the present invention is to provide a composition that provides enhanced healing for the management of ocular infections.

Still another object of the present invention is to provide method of preparation of a composition for the management of ocular infections.

The present invention relates to a composition for treating ocular infections and a method of preparation thereof. More particularly, the present invention relates to a composition comprising vitamin D nanoemulsion and an antimicrobial agent for the management of ocular infections with enhanced healing and a method of preparation of the same.

In an embodiment, the present invention provides a pharmaceutical composition for ocular infection, comprising a nanoemulsion of fat-soluble secosteroid; an antimicrobial agent; and a pharmaceutically acceptable excipient; wherein, said fat-soluble secosteroid is vitamin D; said antimicrobial agent is vancomycin or tobramycin; and said vitamin D acts as a carrier and a catalyst to said antimicrobial agent.

In another embodiment, the present invention provides a pharmaceutical composition for ocular infection, comprising vitamin D in the range of 800 IU-3200 IU; vancomycin or tobramycin in the range of 0.15%-0.5% (w/v); and vitamin D acts as a carrier as well as catalyst to bioactive agent.

In yet another embodiment of the present invention, said vitamin D includes, but not limited to, ergocalciferol, cholecalciferol, 22-dihydroergocalciferol, sitocalciferol, mixture of ergocalciferol and lumisterol and their other derivatives.

In yet another embodiment of the present invention, said vitamin is preferably Vitamin D3 (cholecalciferol).

In yet another embodiment of the present invention, said composition is formulated for controlled drug delivery in a range of 1-15 days.

In yet another embodiment of the present invention, said composition is in form of a solution, suspension, emulsion, nanoparticles, micro emulsion, neosomes, nanoemulsion, ointment, cream, gel, ocular insert or sustained release vehicle.

In yet another embodiment, the present invention provides for encapsulation of vitamin D in an ophthalmic drug-delivery system including liposome, neosomes, microsphere, nanoparticles, nanoemulsion, gel-like protein, collagen, soft contact lenses or intraocular lenses or to adhere such vitamin to the drug-delivery system for direct administration to damaged tissues or sites in the form of an ophthalmic treating agent.

In yet another embodiment, the present invention provides for admixture of vitamin D with at least one viscous base or oil base selected from the group consisting of polyvinyl alcohol, methyl cellulose, hyaluronic acid, chondroitin sulfuric acid, collagen, oils and fats to provide a viscous ophthalmic solution which can be used as an eye drop or an agent directly administered into the aqueous humor.

In yet another embodiment, the present invention provides for a process for preparation of a pharmaceutical composition, comprising the steps of mixing an emulsifier and a surfactant in a pre-defined ratio and stirring for 50-60 minutes to obtain a S-mix; dissolving pre-defined amount of vitamin D in pre-defined amount of a medium chain triglyceride and further adding 1-2% (w/v) of S-mix obtained in step (a) with continuous stirring for 30 minutes obtain a mixture; adding the mixture obtained in step (b) drop wise in water and stirring at a pre-defined speed for 10 minutes to obtain vitamin D nanoemulsion; adding dropwise a pre-defined amount of antimicrobial agent to the vitamin D nanoemulsion obtained in step (c); wherein, the pre-defined ratio of said emulsifier and said surfactant is 2:1; the pre-defined amount of vitamin D is in the range of 0.4-1.6 mg; the pre-defined amount of a medium chain triglyceride is in the range of 0.1-1% (w/v); the pre-defined stirring speed is 800 rpm; and vitamin D acts as a carrier as well as catalyst to bioactive agent thus resulting in enhanced healing.

The present invention provides a composition and method of preparation of the said composition for the management of ocular infections. The composition shows enhanced healing in managing ocular infections. More particularly, the present invention provides a composition comprising of vitamin D nanoemulsion, antimicrobial and pharmaceutically acceptable salt for the management of ocular infections.

The above objects and advantages of the present invention will become apparent from the hereinafter set forth detailed description of the invention, and claims appended herewith.

### DETAILED DESCRIPTION OF THE INVENTION

The present invention now will be described hereinafter with reference to the detailed description, in which some, but not all embodiments of the invention are indicated. Indeed, the invention may be embodied in many different forms and should not be construed as limited to the embodiments set forth herein; rather, these embodiments are provided so that this disclosure will satisfy applicable legal requirements. Like numbers refer to like elements throughout. The present invention is described fully herein with non-limiting embodiments and exemplary experimentation.

In a preferred embodiment, the present invention provides a pharmaceutical composition for ocular infection, comprising a nanoemulsion of fat-soluble secosteroid; an antimicrobial agent; and a pharmaceutically acceptable excipient; wherein, said fat-soluble secosteroid is vitamin D; said antimicrobial agent is vancomycin or tobramycin; and said vitamin D acts as a carrier and a catalyst to said antimicrobial agent.

In another preferred embodiment, the present invention provides a pharmaceutical composition for ocular infection, comprising vitamin D in the range of 800 IU-3200 IU; vancomycin or tobramycin in the range of 0.15%-0.5% (w/v); and vitamin D acts as a carrier as well as catalyst to bioactive agent.

In yet another preferred embodiment, the present invention provides a composition comprising of vitamin D nanoemulsion, an antimicrobial agent and a pharmaceutically acceptable excipient. The composition shows anti-microbial properties with enhanced healing and is effective in the management of ocular infection.

In yet another preferred embodiment of the present invention, said vitamin D includes, but not limited to, ergocalciferol, cholecalciferol, 22-dihydroergocalciferol, sitocalciferol, mixture of ergocalciferol and lumisterol and their other derivatives.

In yet another preferred embodiment of the present invention, said vitamin is preferably Vitamin D3 (cholecalciferol).

In yet another preferred embodiment, the present invention comprises of an antimicrobial agent which allows for treating or preventing multiple conditions or symptoms, simultaneously, which is present in any of the ophthalmic compositional forms described herein including fluid and solid forms, are pharmaceutically active compounds having efficacy in ocular application and which are compatible with the bioactive agent and with the eye. Typically, the additional medicaments include other antibiotics, antivirals, antifungals, anesthetics, anti-inflammatory agents including steroidal and non-steroidal anti-inflammatories, and anti-allergic agents. These other medicaments are generally present in a therapeutically effective amount.

In yet another preferred embodiment, the present invention provides for a pharmaceutically acceptable excipient including, but not limited to, buffering agents, preservatives, coloring agents and stabilizers that have a broad range of functionalities in making formulations in the form of bulking agents, binders, disintegrants, flavors, glidants, lubricants, preservatives, permeation enhancers, solubility enhancers, preservatives and sweeteners, etc. Further, to enhance the drug bioavailability, excipients are used which enhance the stability and increases the solubility of the drug. Solubility enhancing excipients are mainly categorized into three sections called as a polymer, surfactant, and lipid based. However, polymer-based excipients are widely used for solubility enrichment process. The surfactants in surfactant-based excipient facilitate their solubilization. Further, surfactants are able to solubilize the poorly soluble drug molecules by micelle formation or by acting as co-solvents. The combination of lipophilic surfactants, hydrophilic surfactants, water-soluble co-solvents, triglyceride oils, co-surfactants build the efficient and stable self-emulsifying drug delivery system which improve drug solubility and oral absorption.

In yet another preferred embodiment, the antibiotics are selected from a group containing aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; sulfonamides; polymyxin; chloramphenicol; neomycin; paramomomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; comolyn; lodoxamide; levocabastin; naphazoling; antazoline; and pheniramimane.

The composition of the present invention is in the form of a solution, suspension, emulsion, nanoparticles, micro emulsion, neosomes, nanoemulsion, ointment, cream, gel, or sustained release vehicle, such as an ocular insert. Alternatively, it is also possible to encapsulate active vitamin D in an ophthalmic drug-delivery system such as liposome, microsphere, gel-like protein, collagen, soft contact lenses for treatment or intraocular lenses or to adhere such vitamin to the drug-delivery system to thus directly administer or deliver active vitamin D to damaged tissues or sites in the form of an ophthalmic treating agent.

The lacrymal fluid is viscous and oil-soluble. Therefore, active vitamin D is preferably be admixed with at least one viscous base or oil base selected from the group consisting of for instance, polyvinyl alcohol, methyl cellulose, hyaluronic acid, chondroitin sulfuric acid, collagen, oils and fats to provide a viscous ophthalmic solution which is used as an eye drop or an agent directly administered into the aqueous humor.

In yet another preferred embodiment, the present invention provides for a process for preparation of a pharmaceutical composition, comprising the steps of mixing an emulsifier and a surfactant in a pre-defined ratio and stirring for 50-60 minutes to obtain a S-mix; dissolving pre-defined amount of vitamin D in pre-defined amount of a medium chain triglyceride and further adding pre-defined amount of S-mix obtained in step (a) with continuous stirring for 30 minutes obtain a mixture; adding the mixture obtained in step (b) drop wise in water and stirring at a pre-defined speed for 10 minutes to obtain vitamin D nanoemulsion; adding dropwise a pre-defined amount of antimicrobial agent to the vitamin D nanoemulsion obtained in step (c); wherein, the pre-defined ratio of an emulsifier and a surfactant is 2:1; the pre-defined amount of S-mix is 1-2% (w/v); the pre-defined amount of a medium chain triglyceride is in the range of 0.1-1% (w/v); and vitamin D acts as a carrier as well as catalyst to bioactive agent thus resulting in enhanced healing.

In yet another preferred embodiment of the present invention, the pre-defined amount of vitamin D is in the range of 0.4-1.6 mg.

In yet another preferred embodiment of the present invention, the pre-defined amount of said antimicrobial agent is 0.15-0.3% (w/v).

In yet another embodiment of the present invention, the pre-defined stirring speed of the mixture in step (c) is 800 rpm.

In yet another embodiment of the present invention, the emulsifier is a non-ionic emulsifier, polyoxyethylene sorbitan monooleate (Tween 80) and the surfactant is a low-molecular-weight grade of polyethylene glycol, polyethylene glycol 400 (PEG 400). Tween 80 is a viscous, water-soluble yellow liquid used in pharmaceutical composition and PEG 400 is a clear, colourless, viscous liquid and owing to its low toxicity, PEG 400 is widely used in a variety of pharmaceutical formulations. The medium chain triglyceride is Labrafac oil, that is an oily vehicle and solubilizer of lipophilic API for oral, topical and parenteral lipid based formulations.

In still another embodiment, the present invention provides for vitamin D as a carrier for the drugs, preferably antibiotics including, but not limited to, aminoglycosides, Beta-lactam agents, Quinolones.

The following examples and advantages of the present invention are provided for the purpose of illustration only and are not intended to limit the scope of the present invention.

### Example 1

### Method of Preparation of Vitamin D Nanoemulsion

Preparing a S-mix by mixing Tween 80 and PEG 400 in ratio 2:1 respectively and keeping it on stirring for one hour. Further, dissolving 1 mg vitamin D in 0.5% (w/v) of Labrafac Oil and then adding 1% (w/v) of prepared S-mix with continuous stirring for 30 minutes and adding the prepared mixture drop wise in water with stirring speed of 800 rpm for 10 minutes to obtain said composition. Table 1 shows the composition used in preparing vitamin D nanoemulsion.

TABLE 1
Composition for preparing Vitamin D nanoemulsion

| S. No. | Material | Quantity |
|---|---|---|
| 1 | S-mix (Tween 80 + PEG 400 = 2:1) | 1% |
| 2 | Labrafac | 0.5% |
| 3 | Vitamin D | 2000 IU/mg |
| 4 | Water | Qs. to volume |

### Example 2

### Compositions for Managing Ocular Infections

In exemplary procedures for managing ocular infections, the S-mix was prepared by mixing Tween 80 and PEG 400 in a desired ratio with predefined amount of vitamin D, pre-defined amount of Labrafac oil and pre-defined amount of antimicrobial agent. Table 2 shows the different compositions used for managing ocular infections.

TABLE 2
Different compositions for managing ocular infections

| | | Vancomycin/ | | |
|---|---|---|---|---|
| S. | Vitamin-D | Tobramycin | Smix (%):Tween | Labrafac |
| No. | (IU/mg) | (%) | 80 and PEG400 | (%) |
| 1. | 800 IU/0.4 mg | 0.15% | 1% (2:1) | 0.1% |
| 2. | 1000 IU/0.5 mg | 0.2% | 1% (2:1) | 0.11% |
| 3. | 1200 IU/0.6 mg | 0.25% | 1% (2:1) | 0.13% |
| 4. | 1400 IU/0.7 mg | 0.27% | 1% (1:1) | 0.16% |
| 5. | 1600 IU/0.8 mg | 0.28% | 1% (2:1) | 0.2% |
| 6. | 1700 IU/0.85 mg | 0.29% | 1% (2:1) | 0.25% |
| 7. | 1800 IU/0.90 mg | 0.3% | 1% (2:1) | 0.3% |
| 8. | 1950 IU/0.975 mg | 0.3% | 1% (2:1) | 0.39% |
| 9. | 2000 IU/1.0 mg | 0.3% | 1% (2:1) | 0.5% |
| 10. | 2050 IU/1.050 mg | 0.3% | 1% (1:2) | 0.55% |
| 11. | 2100 IU/1.01 mg | 0.32% | 2% (2:1) | 0.59% |
| 12. | 2200 IU/1.1 mg | 0.34% | 1% (2:1) | 0.6% |
| 13. | 2300 IU/1.15 mg | 0.38% | 1% (2:1) | 0.65% |
| 14. | 2400 IU/1.20 mg | 0.40% | 1% (2:1) | 0.65% |
| 15. | 2500 IU/1.25 mg | 0.4% | 2% (2:1) | 0.70% |
| 16. | 2600 IU/1.30 mg | 0.42% | 1% (2:1) | 0.765% |
| 17. | 2800 IU/1.40 mg | 0.45% | 1% (2:1) | 0.8% |
| 18. | 2900 IU/1.45 mg | 0.48% | 1% (2:1) | 0.87% |
| 19. | 3000 IU/1.5 mg | 0.5% | 1% (2:1) | 0.9% |
| 20. | 3200 IU/1.6 mg | 0.5% | 2% (2:1) | 1.0% |

Therefore, the present invention provides a pharmaceutical composition and method of preparation of the said composition for the management of ocular infections. The composition shows enhanced healing in managing ocular infections. More particularly, the present invention provides a composition comprising of vitamin D nanoemulsion, antimicrobial and pharmaceutically acceptable salt for the management of ocular infections.

Many modifications and other embodiments of the invention set forth herein will readily occur to one skilled in the art to which the invention pertain having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A pharmaceutical composition for ocular infection, comprising:
a nanoemulsion of fat-soluble secosteroid;
an antimicrobial agent; and
a pharmaceutically acceptable excipient;
wherein,
said fat-soluble secosteroid is vitamin D;
said antimicrobial agent is vancomycin or tobramycin; and
said vitamin D acts as a carrier and a catalyst to said antimicrobial agent.

2. The composition as claimed in claim 1, wherein the concentration of said vitamin D is in the range of 800-3200 IU.

3. The composition as claimed in claim 1, wherein the concentration of said antimicrobial agent is 0.15-0.5% (w/v).

4. The composition as claimed in claim 1, wherein said composition is formulated for controlled drug delivery in a range of 1-15 days.

5. The composition as claimed in claim 1, wherein said vitamin D is ergocalciferol, cholecalciferol, 22-dihydroergocalciferol, sitocalciferol, mixture of ergocalciferol and lumisterol and other derivatives.

6. The composition as claimed in claim 1, wherein said vitamin D is preferably Vitamin D3 (cholecalciferol).

7. The composition as claimed in claim 1, wherein said composition is in form of a solution, suspension, emulsion, nanoparticles, micro emulsion, neosomes, nanoemulsion, ointment, cream, gel, ocular insert or sustained release vehicle.

8. The composition as claimed in claim 1, wherein said pharmaceutical excipient is selected from the group of buffering agents, preservatives, colouring agents or stabilisers.

9. The composition as claimed in claim 1, wherein vitamin D is encapsulated in drug-delivery system including liposome, neosomes, microsphere, nanoparticles, nano emulsion, gel-like protein, collagen, soft contact lenses, ocuserts, or intraocular lenses.

10. The composition as claimed in claim 1, wherein active vitamin D is admixed with at least one viscous base or oil base selected from the group of polyvinyl alcohol, methyl cellulose, hyaluronic acid, chondroitin sulfuric acid, collagen, oils or fats.

11. A process for preparation of a pharmaceutical composition for ocular infection, comprising the steps of:
a) mixing an emulsifier and a surfactant in a pre-defined ratio and stirring for 50-60 minutes to obtain a S-mix;
b) dissolving pre-defined amount of vitamin D in pre-defined amount of a medium chain triglyceride and adding pre-defined amount of S-mix obtained in step (a) with continuous stirring for 30 minutes to obtain a mixture;
c) adding the mixture obtained in step (b) drop wise in water and stirring at a pre-defined stirring speed for 10 minutes to obtain vitamin D nanoemulsion; and
d) adding dropwise a pre-defined amount of antimicrobial agent to the vitamin D nanoemulsion obtained in step (c);
wherein,
the pre-defined ratio of said emulsifier and said surfactant is 2:1;
the pre-defined amount of S-mix is in the range of 1-2% (w/v);
the pre-defined amount of said medium chain triglyceride is in the range of 0.1-1% (w/v); and
the vitamin D acts as a carrier as well as catalyst to said antimicrobial agent.

12. The process as claimed in claim 11, wherein said emulsifier is polyoxyethylene sorbitan monooleate (Tween 80).

13. The process as claimed in claim 11, wherein said surfactant is Polyethylene glycol 400 (PEG 400).

14. The process as claimed in claim 11, wherein said medium chain triglyceride is Labrafac oil.

15. The process as claimed in claim 11, wherein the pre-defined amount of vitamin D is in the range of 0.4-1.6 mg.

16. The process as claimed in claim 11, wherein the pre-defined amount of said antimicrobial agent is 0.15-0.5% (w/v).

17. The process as claimed in claim 11, wherein the pre-defined stirring speed of the mixture in step (c) is 800 rpm.
